(19) United States
(12) Patent Application Publication
(10) Pub. No.: US 2025/0327127 A1
(43) Pub. Date: Oct. 23, 2025

(54) SMALL MOLECULE TREATMENT OF MTRES1 RELATED DISEASES AND DISORDERS

(71) Applicant: Empirico Inc., San Diego, CA (US)

(72) Inventors: Omri GOTTESMAN, San Diego, CA (US); Emma BRANDT, Madison, WI (US); Shannon BRUSE, San Diego, CA (US); Paul BUSKE, Middleton, WI (US); Brian CAJES, San Diego, CA (US); David JAKUBOSKY, San Diego, CA (US); David LEWIS, Madison, WI (US); Gregory MCINNES, San Diego, CA (US); David ROZEMA, Cross Plains, WI (US); John VEKICH, San Diego, CA (US)

(21) Appl. No.: 19/122,140

(22) PCT Filed: Oct. 23, 2023
(86) PCT No.: PCT/US2023/077527

§ 371 Date: Apr. 17, 2025

Related U.S. Application Data
(60) Provisional application No. 63/419,208, filed on Oct. 25, 2022.

(51) Int. Cl. C12Q 1/6883; A61K 45/06; A61P 25/28

(52) U.S. Cl. CPC C12Q 1/6883; A61K 45/06; A61P 25/28; C12Q 2600/106; C12Q 2600/158

(57) ABSTRACT

Disclosed herein are compositions comprising a small molecule that targets MTRES1. Also provided herein are methods of treating conditions associated with MTRES1 gene mutations that include providing a small molecule that targets MTRES1 in a subject.

### CROSS-REFERENCE

This application claims the benefit of U.S. Provisional Application No. 63/419,208 filed on Oct. 25, 2022, which is incorporated herein by reference in its entirety.

### BACKGROUND

Neurological disorders are a common problem, particularly in the older population. Improved therapeutics are needed for treating these disorders.

### SUMMARY

Disclosed herein, in some embodiments, are compositions comprising a small molecule that targets MTRES1 and when administered to a subject in an effective amount modulates the activity, function, or binding of central nervous system (CNS) MTRES1. In some embodiments, the activity, function or binding of CNS MTRES1 is inhibited. In some embodiments, the activity, function, or binding of CNS MTRES1 is inhibited by about 10% or more, as compared to prior to administration. Disclosed herein, in some embodiments, are compositions comprising a small molecule that targets MTRES1 and when administered to a subject in an effective amount increases cognitive function or slows cognitive decline. In some embodiments, the cognitive function is increased by about 10% or more, as compared to prior to administration. In some embodiments, the cognitive decline is slowed by about 10% or more, as compared to prior to administration. Disclosed herein, in some embodiments, are compositions comprising a small molecule that targets MTRES1 and when administered to a subject in an effective amount decreases a marker of neurodegeneration. In some embodiments, the marker of neurodegeneration comprises a central nervous system (CNS) or cerebrospinal fluid (CSF) marker of neurodegeneration. In some embodiments, the marker of neurodegeneration comprises a measurement of central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. In some embodiments, the marker of neurodegeneration is decreased by about 10% or more, as compared to prior to administration. Disclosed herein, in some embodiments, are methods of treating a subject having a neurological disorder, comprising administering an effective amount of a composition described herein to the subject. In some embodiments, the neurological disorder comprises dementia, Alzheimer's disease, delirium, cognitive decline, vascular dementia, or Parkinson's disease.

In certain aspects, disclosed herein is a method of treating a subject having a neurological disorder, comprising administering an effective amount of the composition disclosed herein to the subject. In some embodiments, disclosed herein is a method of treating a subject having a neurological disorder or who is at risk for developing the neurological disorder, the method comprising evaluating a subject's risk for developing a neurological disorder and administering an effective amount of the composition disclosed herein to the subject. In some embodiments, the subject has a genotype at risk for developing Alzheimer's disease or dementia. In some embodiments, the subject is a heterozygous or homozygous carrier of APOE4. In some embodiments, the subject is a heterozygous or homozygous carrier of MTRES1 rs117058816-G (c.3+1G). In some embodiments, evaluating a subject's risk for developing a neurological disorder comprises calculating a polygenic risk score for developing Alzheimer's disease or dementia. In some embodiments, the subject has a polygenic risk score in the 40th percentile or higher, which is indicative of a high risk for developing Alzheimer's disease or dementia. In some embodiments, the subject has a polygenic risk score in the 20th percentile or higher, which is indicative of a high risk for developing Alzheimer's disease or dementia. In some embodiments, calculating a polygenic risk score comprises providing genomic data comprising one or more genotypes of the subject, wherein the one or more genotypes is associated with a high risk for developing Alzheimer's disease or dementia.

### BRIEF DESCRIPTION OF THE DRAWINGS

FIG. 1A is an image of a western blot of MTRES1 protein in the wildtype and MTRES1 c.3+1G>A minigene expression constructs. Lane 1: ladder (bands for 38, 31, 24, and 17 kDa are indicated); lane 2: Empty vector; lane 3: wild type (WT); lane 4: c.3+1G>A.

FIG. 1B is a plot quantifying MTRES1 western blot data in the wildtype and MTRES1 c.3+1G>A minigene expression constructs. Left bar: wild type (WT); right bar: c.3+1G>A. The y-axis is labeled normalized to total protein from 0 to 4 at 1 unit intervals.

FIG. 2 is a plot of MTRES1 mRNA qPCR data in the wildtype and MTRES1 c.3+1G>A minigene expression constructs. Left bar: wild type (WT); right bar: c.3+1G>A. The y-axis is labeled fold change from empty vector from 0 to 100 at 20 unit intervals.

FIG. 3 is an image of a western blot of MTRES1 protein in the CRISPR-engineered wildtype, MTRES1 c.3+1G>A knock-in cells, and MTRES1 knock-out cell lines. Left column: whole cell lysate; right column: Mito fraction. Rows (top to bottom): wild type (WT); c.3+1G>A; KO.

FIG. 4 is a plot of MTRES1 mRNA qPCR data in the CRISPR-engineered wildtype, MTRES1 c.3+1G>A knock-in cells, and MTRES1 knock-out cell lines. Left bar: wild type (WT); middle bar: c.3+1G>A; right bar: KO. The y-axis is labeled fold change from 0.0 to 1.5 at 0.5 unit intervals.

### DETAILED DESCRIPTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, brain) and a relevant indication.

The MTRES1 gene is located on chromosome 6, and encodes mitochondrial transcription rescue factor 1 (MTRES1), also known as chromosome 6 open reading frame 203 (C6orf203). The MTRES1 gene may also be referred to as the (6orf203 gene. MTRES1 may include 240 amino acids. MTRES1 may include 245 amino acids. MTRES1 may be expressed in neural cells. MTRES1 may be cytoplasmic or intracellular. MTRES1 may be localized in mitochondria within the cell. MTRES1 may be involved in mitochondrial transcription regulation. MTRES1 may be involved in mitochondrial translation regulation. An example of a MTRES1 amino acid sequence, and further description of MTRES1 is included at uniprot.org under accession no. Q9P0P8 (last modified Oct. 1, 2000).

Here it is shown that loss of function MTRES1 variants may protect against neurological diseases. For example, a loss of function MTRES1 variant was associated with protective associations against Alzheimer's disease, family history of Alzheimer's disease, dementia, vascular dementia, anticholinesterase medication use, and delirium. Therefore, inhibition of MTRES1 may serve as a therapeutic for treatment of a neurological disorder such as dementia, Alzheimer's disease, delirium, cognitive decline, vascular dementia, or Parkinson's disease.

Disclosed herein are compositions comprising a small molecule that targets MTRES1. Where inhibition or targeting of MTRES1 is disclosed, it is contemplated that some embodiments may include inhibiting or targeting a MTRES1 protein or MTRES1 RNA. For example, by inhibiting or targeting a MTRES1 protein using a small molecule described herein, the MTRES1 protein may be inhibited. For instance, a small molecule may interfere with the ability of the MTRES1 protein to bind RNA. Also provided herein are methods of treating a neurological disorder by providing a small molecule that targets MTRES1 to a subject in need thereof.

### I. Compositions

Disclosed herein, in some embodiments, are compositions comprising a small molecule. In some embodiments, the composition comprises a small molecule that targets MTRES1 mRNA or protein. In some embodiments, the composition consists of a small molecule that targets MTRES1 mRNA or protein. In some embodiments, the small molecule modulates MTRES1 activity in the subject. In some embodiments, the small molecule reduces MTRES1 activity in the subject. In some embodiments, a composition described herein is used in a method of treating a disorder in a subject in need thereof. Some embodiments relate to a composition comprising a small molecule for use in a method of treating a disorder as described herein. Some embodiments relate to use of a composition comprising a small molecule, in a method of treating a disorder as described herein.

Some embodiments include a composition comprising a small molecule that targets MTRES1 and when administered to a subject in an effective amount alters MTRES1 activity levels in a cell, fluid or tissue. Some embodiments include a composition comprising a small molecule that targets MTRES1 and when administered to a subject in an effective amount decreases MTRES1 activity levels in a cell, fluid or tissue. In some embodiments, the composition comprises a small molecule that targets MTRES1 and when administered to a subject in an effective amount decreases MTRES1 activity levels in a cell or tissue. In some embodiments, the cell is a neural cell such as a central nervous system (CNS) cell. Some examples of CNS cells include neurons, glia, microglia, astrocytes, or oligodendrocytes. In some embodiments, the tissue is CNS or brain tissue. In some embodiments, the MTRES1 activity levels are decreased by about 2.5% or more, about 5% or more, or about 7.5% or more, as compared to prior to administration. In some embodiments, the MTRES1 activity levels are decreased by about 10% or more, as compared to prior to administration. In some embodiments, the MTRES1 activity levels are decreased by about 20% or more, about 30% or more, about 40% or more, about 50% or more, about 60% or more, about 70% or more, about 80% or more, about 90% or more, or about 100%, as compared to prior to administration. In some embodiments, the MTRES1 activity levels are decreased by no more than about 2.5%, no more than about 5%, or no more than about 7.5%, as compared to prior to administration. In some embodiments, the MTRES1 activity levels are decreased by no more than about 10%, as compared to prior to administration. In some embodiments, the MTRES1 activity levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MTRES1 activity levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount modulates MTRES1 mRNA or protein levels in a cell, fluid or tissue. In some embodiments, the composition comprises a small molecule that targets MTRES1 and when administered to a subject in an effective amount decreases MTRES1 mRNA or protein levels in a cell, fluid or tissue. In some embodiments, the cell is a neural cell such as a central nervous system (CNS) cell. Some examples of CNS cells include neurons, glia, microglia, astrocytes, or oligodendrocytes. In some embodiments, the tissue is CNS or brain tissue. In some embodiments, the MTRES1 mRNA or protein levels are decreased by about 2.5% or more, about 5% or more, or about 7.5% or more, as compared to prior to administration. In some embodiments, the MTRES1 mRNA or protein levels are decreased by about 10% or more, as compared to prior to administration. In some embodiments, the MTRES1 mRNA or protein levels are decreased by about 20% or more, about 30% or more, about 40% or more, about 50% or more, about 60% or more, about 70% or more, about 80% or more, about 90% or more, or about 100%, as compared to prior to administration. In some embodiments, the MTRES1 mRNA or protein levels are decreased by no more than about 2.5%, no more than about 5%, or no more than about 7.5%, as compared to prior to administration. In some embodiments, the MTRES1 mRNA or protein levels are decreased by no more than about 10%, as compared to prior to administration. In some embodiments, the MTRES1 mRNA or protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the MTRES1 mRNA or protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount diminishes a neurological disorder phenotype. The neurological disorder disease may include dementia, Alzheimer's disease, delirium, cognitive decline, vascular dementia, or Parkinson's disease. In some embodiments, the neurological disorder phenotype is decreased by about 2.5% or more, about 5% or more, or about 7.5% or more, as compared to prior to administration. In some embodiments, the neurological disorder phenotype is decreased by about 10% or more, as compared to prior to administration. In some embodiments, the neurological disorder phenotype is decreased by about 20% or more, about 30% or more, about 40% or more, about 50% or more, about 60% or more, about 70% or more, about 80% or more, about 90% or more, or about 100%, as compared to prior to administration. In some embodiments, the neurological disorder phenotype is decreased by no more than about 2.5%, no more than about 5%, or no more than about 7.5%, as compared to prior to administration. In some embodiments, the neurological disorder phenotype is decreased by no more than about 10%, as compared to prior to administration. In some embodiments, the neurological disorder phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the neurological disorder phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount enhances a protective phenotype against a neurological disorder in the subject. The neurological disorder may include dementia, Alzheimer's disease, delirium, cognitive decline, vascular dementia, or Parkinson's disease. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises a small molecule that targets MTRES1 and when administered to a subject in an effective amount decreases a marker of neurodegeneration in the subject. Some example markers of neurodegeneration may include central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. In some embodiments, the marker of neurodegeneration is decreased by about 2.5% or more, about 5% or more, or about 7.5% or more, as compared to prior to administration. In some embodiments, the marker of neurodegeneration is decreased by about 10% or more, as compared to prior to administration. In some embodiments, the marker of neurodegeneration is decreased by about 20% or more, about 30% or more, about 40% or more, about 50% or more, about 60% or more, about 70% or more, about 80% or more, about 90% or more, or about 100%, as compared to prior to administration. In some embodiments, the marker of neurodegeneration is decreased by no more than about 2.5%, no more than about 5%, or no more than about 7.5%, as compared to prior to administration. In some embodiments, the marker of neurodegeneration is decreased by no more than about 10%, as compared to prior to administration. In some embodiments, the marker of neurodegeneration is decreased by no more than about 20%, no more than about 30%, no more than about 40%, no more than about 50%, no more than about 60%, no more than about 70%, no more than about 80%, or no more than about 90%, as compared to prior to administration. In some embodiments, the marker of neurodegeneration is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases central nervous system (CNS) amyloid plaques in the subject. In some embodiments, the CNS amyloid plaques are decreased by about 2.5% or more, about 5% or more, or about 7.5% or more, as compared to prior to administration. In some embodiments, the CNS amyloid plaques are decreased by about 10% or more, as compared to prior to administration. In some embodiments, the CNS amyloid plaques are decreased by about 20% or more, about 30% or more, about 40% or more, about 50% or more, about 60% or more, about 70% or more, about 80% or more, about 90% or more, or about 100%, as compared to prior to administration. In some embodiments, the CNS amyloid plaques are decreased by no more than about 2.5%, no more than about 5%, or no more than about 7.5%, as compared to prior to administration. In some embodiments, the CNS amyloid plaques are decreased by no more than about 10%, as compared to prior to administration. In some embodiments, the CNS amyloid plaques are decreased by no more than about 20%, no more than about 30%, no more than about 40%, no more than about 50%, no more than about 60%, no more than about 70%, no more than about 80%, or no more than about 90%, as compared to prior to administration. In some embodiments, the CNS amyloid plaques are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases central nervous system (CNS) tau accumulation in the subject. In some embodiments, the CNS tau accumulation is decreased by about 2.5% or more, about 5% or more, or about 7.5% or more, as compared to prior to administration. In some embodiments, the CNS tau accumulation is decreased by about 10% or more, as compared to prior to administration. In some embodiments, the CNS tau accumulation is decreased by about 20% or more, about 30% or more, about 40% or more, about 50% or more, about 60% or more, about 70% or more, about 80% or more, about 90% or more, or about 100%, as compared to prior to administration. In some embodiments, the CNS tau accumulation is decreased by no more than about 2.5%, no more than about 5%, or no more than about 7.5%, as compared to prior to administration. In some embodiments, the CNS tau accumulation is decreased by no more than about 10%, as compared to prior to administration. In some embodiments, the CNS tau accumulation is decreased by no more than about 20%, no more than about 30%, no more than about 40%, no more than about 50%, no more than about 60%, no more than about 70%, no more than about 80%, or no more than about 90%, as compared to prior to administration. In some embodiments, the CNS tau accumulation is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases cerebrospinal fluid (CSF) beta-amyloid 42 in the subject. In some embodiments, the CSF beta-amyloid 42 is decreased by about 2.5% or more, about 5% or more, or about 7.5% or more, as compared to prior to administration. In some embodiments, the CSF beta-amyloid 42 is decreased by about 10% or more, as compared to prior to administration. In some embodiments, the CSF beta-amyloid 42 is decreased by about 20% or more, about 30% or more, about 40% or more, about 50% or more, about 60% or more, about 70% or more, about 80% or more, about 90% or more, or about 100%, as compared to prior to administration. In some embodiments, the CSF beta-amyloid 42 is decreased by no more than about 2.5%, no more than about 5%, or no more than about 7.5%, as compared to prior to administration. In some embodiments, the CSF beta-amyloid 42 is decreased by no more than about 10%, as compared to prior to administration. In some embodiments, the CSF beta-amyloid 42 is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF beta-amyloid 42 is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases cerebrospinal fluid (CSF) tau in the subject. In some embodiments, the CSF tau is decreased by about 2.5% or more, about 5% or more, or about 7.5% or more, as compared to prior to administration. In some embodiments, the CSF tau is decreased by about 10% or more, as compared to prior to administration. In some embodiments, the CSF tau is decreased by about 20% or more, about 30% or more, about 40% or more, about 50% or more, about 60% or more, about 70% or more, about 80% or more, about 90% or more, or about 100%, as compared to prior to administration. In some embodiments, the CSF tau is decreased by no more than about 2.5%, no more than about 5%, or no more than about 7.5%, as compared to prior to administration. In some embodiments, the CSF tau is decreased by no more than about 10%, as compared to prior to administration. In some embodiments, the CSF tau is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF tau is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases cerebrospinal fluid (CSF) tau in the subject. In some embodiments, the CSF phospho-tau is decreased by about 2.5% or more, about 5% or more, or about 7.5% or more, as compared to prior to administration. In some embodiments, the CSF phospho-tau is decreased by about 10% or more, as compared to prior to administration. In some embodiments, the CSF phospho-tau is decreased by about 20% or more, about 30% or more, about 40% or more, about 50% or more, about 60% or more, about 70% or more, about 80% or more, about 90% or more, or about 100%, as compared to prior to administration. In some embodiments, the CSF phospho-tau is decreased by no more than about 2.5%, no more than about 5%, or no more than about 7.5%, as compared to prior to administration. In some embodiments, the CSF phospho-tau is decreased by no more than about 10%, as compared to prior to administration. In some embodiments, the CSF phospho-tau is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF phospho-tau is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases cerebrospinal fluid (CSF) alpha-synuclein in the subject. In some embodiments, the CSF alpha-synuclein is decreased by about 2.5% or more, about 5% or more, or about 7.5% or more, as compared to prior to administration. In some embodiments, the CSF alpha-synuclein is decreased by about 10% or more, as compared to prior to administration. In some embodiments, the CSF alpha-synuclein is decreased by about 20% or more, about 30% or more, about 40% or more, about 50% or more, about 60% or more, about 70% or more, about 80% or more, about 90% or more, or about 100%, as compared to prior to administration. In some embodiments, the CSF alpha-synuclein is decreased by no more than about 2.5%, no more than about 5%, or no more than about 7.5%, as compared to prior to administration. In some embodiments, the CSF alpha-synuclein is decreased by no more than about 10%, as compared to prior to administration. In some embodiments, the CSF alpha-synuclein is decreased by no more than about 20%, no more than about 30%, no more than about 40%, no more than about 50%, no more than about 60%, no more than about 70%, no more than about 80%, or no more than about 90%, as compared to prior to administration. In some embodiments, the CSF alpha-synuclein is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount decreases Lewy bodies in the subject. In some embodiments, the Lewy bodies are decreased by about 2.5% or more, about 5% or more, or about 7.5% or more, as compared to prior to administration. In some embodiments, the Lewy bodies are decreased by about 10% or more, as compared to prior to administration. In some embodiments, the Lewy bodies are decreased by about 20% or more, about 30% or more, about 40% or more, about 50% or more, about 60% or more, about 70% or more, about 80% or more, about 90% or more, or about 100%, as compared to prior to administration. In some embodiments, the Lewy bodies are decreased by no more than about 2.5%, no more than about 5%, or no more than about 7.5%, as compared to prior to administration. In some embodiments, the Lewy bodies are decreased by no more than about 10%, as compared to prior to administration. In some embodiments, the Lewy bodies are decreased by no more than about 20%, no more than about 30%, no more than about 40%, no more than about 50%, no more than about 60%, no more than about 70%, no more than about 80%, or no more than about 90%, as compared to prior to administration. In some embodiments, the Lewy bodies are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a small molecule that targets MTRES1 and when administered to a subject in an effective amount increases cognitive function. In some embodiments, the cognitive function is increased by about 2.5% or more, about 5% or more, or about 7.5% or more, as compared to prior to administration. In some embodiments, the cognitive function is increased by about 10% or more, as compared to prior to administration. In some embodiments, the cognitive function is increased by about 20% or more, about 30% or more, about 40% or more, about 50% or more, about 60% or more, about 70% or more, about 80% or more, about 90% or more, or about 100% or more, as compared to prior to administration. In some embodiments, the cognitive function is increased by about 200% or more, about 300% or more, about 400% or more, about 500% or more, about 600% or more, about 700% or more, about 800% or more, about 900% or more, or about 1000% or more, as compared to prior to administration. In some embodiments, the cognitive function is increased by no more than about 2.5%, no more than about 5%, or no more than about 7.5%, as compared to prior to administration. In some embodiments, the cognitive function is increased by no more than about 10%, as compared to prior to administration. In some embodiments, the cognitive function is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the cognitive function is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the cognitive function is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

Some embodiments include a composition comprising a small molecule and when administered to a subject in an effective amount alters the activity, binding, or function of MTRES1 mRNA or protein in a cell, fluid or tissue. Some embodiments include a composition comprising a small molecule and when administered to a subject in an effective amount inhibits the activity, binding, or function of MTRES1 mRNA or protein in a cell, fluid or tissue. In some embodiments, the composition comprises a small molecule and when administered to a subject in an effective amount inhibits the activity, binding, or function of MTRES1 mRNA or protein in a cell, fluid or tissue. In some embodiments, the cell is a neural cell such as a central nervous system (CNS) cell.

Some examples of CNS cells include neurons, glia, microglia, astrocytes, or oligodendrocytes. In some embodiments, the tissue is CNS or brain tissue. In some embodiments, the activity, binding, or function of MTRES1 mRNA or protein is decreased by about 2.5% or more, about 5% or more, or about 7.5% or more, as compared to prior to administration. In some embodiments, the activity, binding, or function of MTRES1 mRNA or protein is decreased by about 10% or more, as compared to prior to administration. In some embodiments, the activity, binding, or function of MTRES1 mRNA or protein is decreased by about 20% or more, about 30% or more, about 40% or more, about 50% or more, about 60% or more, about 70% or more, about 80% or more, about 90% or more, or about 100%, as compared to prior to administration. In some embodiments, the activity, binding, or function of MTRES1 mRNA or protein is decreased by no more than about 2.5%, no more than about 5%, or no more than about 7.5%, as compared to prior to administration. In some embodiments, the activity, binding, or function of MTRES1 mRNA or protein is decreased by no more than about 10%, as compared to prior to administration. In some embodiments, the activity, binding, or function of MTRES1 mRNA or protein is decreased by no more than about 20%, no more than about 30%, no more than about 40%, no more than about 50%, no more than about 60%, no more than about 70%, no more than about 80%, or no more than about 90%, as compared to prior to administration. In some embodiments, activity, binding, or function of MTRES1 mRNA or protein is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

### A. Small Molecule Modulators of MTRES1

Provided herein are small molecule modulators of MTRES1 that work through a variety of mechanisms. In some embodiments, MTRES1 is modulated by blocking or altering its RNA binding ability. In some embodiments, the small molecule modulators of MTRES1 are small molecule inhibitors of MTRES1. In some embodiments, the small molecule inhibitor of MTRES1 inhibits MTRES1 activity. In some embodiments, the small molecule inhibitor of MTRES1 inhibits MTRES1 function. In some embodiments, the small molecule inhibitor of MTRES1 inhibits MTRES1 binding. In some embodiments, the small molecule inhibitor of MTRES1 activity inhibits the function, binding, or structure of MTRES1.

An example of a small molecule is an organic compound having a molecular weight of less than 900 Daltons. The molecular weight may be below 2500 Daltons, below 2250 Daltons, below 2000 Daltons, below 1750 Daltons, below 1500 Daltons, or below 1250 Daltons. The ligand may have a molecular weight below 1000 Daltons, below 900 Daltons, below 800 Daltons, below 700 Daltons, below 600 Daltons, or below 500 Daltons. The molecular weight may be greater than 2500 Daltons, greater than 2250 Daltons, greater than 2000 Daltons, greater than 1750 Daltons, greater than 1500 Daltons, or greater than 1250 Daltons. The ligand may have a molecular weight greater than 1000 Daltons, greater than 900 Daltons, greater than 800 Daltons, greater than 700 Daltons, greater than 600 Daltons, or greater than 500 Daltons. The compound may be synthetic.

In some embodiments, the inhibitor of MTRES1 comprises 2-hydroxypropanoic acid, aflatoxin B1, aflatoxin M1, all-trans-retinoic acid, aristolochic acid, bisphenol A, copper (II) sulfate, cyclosporin A, gentamycin, methylmercury chloride, potassium chromate, rac-lactic acid, sunitinib, or thioacetamide. In some embodiments, the small molecule is conjugated to an antibody or antigen-binding fragment thereof. In some embodiments, the antibody or antigen-binding fragment thereof binds MTRES1.

In some embodiments, the inhibitor of MTRES1 comprises an MTRES1 degrader such as a proteolysis targeting chimera (PROTAC). The degrader may include a heterobifunctional compound including a MTRES1 binding moiety and a degradation tag. The degradation tag may include a ubiquitin ligase binder such as a VHL or cereblon binder. The degradation tag and MTRES1 binding moiety may be connected through a linker.

### B. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In some embodiments, the composition is formulated to cross the blood brain barrier. In some embodiments, the composition is formulated for central nervous system (CNS) delivery. In some embodiments, the composition includes a lipophilic compound. The lipophilic compound may be useful for crossing the blood brain barrier or for CNS delivery.

### II. Methods and Uses

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

### A. Disorders

Some embodiments of the methods described herein include treating a disorder in a subject in need thereof. In some embodiments, the disorder is a neurological disorder. Non-limiting examples of neurological disorders include dementia, Alzheimer's disease, delirium, cognitive decline, vascular dementia, or Parkinson's disease. In some embodiments, the neurological disorder includes cognitive decline. In some embodiments, the neurological disorder includes delirium. In some embodiments, the neurological disorder includes dementia. In some embodiments, the neurological disorder includes vascular dementia. In some embodiments, the neurological disorder includes Alzheimer's disease. In some embodiments, the neurological disorder includes Parkinson's disease. The neurological disorder may include a neurodegenerative disease. The neurological disorder may be characterized by protein aggregation.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female.

In some embodiments, the subject is an adult (e.g. at least 18 years old). In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

### C. Genotyping

Disclosed herein, in some embodiments, are systems, methods and kits for detecting one or more genotypes. In some embodiments, the genotypes described herein are detected using suitable genotyping devices (e.g., array, sequencing). In some instances, a sample is obtained from the subject or patient indirectly or directly. In some instances, the sample may be obtained by the subject. In other instances, the sample may be obtained by a healthcare professional, such as a nurse or physician. The sample may be derived from virtually any biological fluid or tissue containing genetic information, such as blood. Methods disclosed herein for detecting a genotype in a sample from a subject comprise analyzing the genetic material in the sample to detect at least one of a presence, an absence, and a quantity of a nucleic acid sequence encompassing the genotype of interest.

In some embodiments, the genotype is a genotype at risk for developing Alzheimer's disease or dementia. In some embodiments, the subject is a heterozygous carrier of APOE4. In some embodiments, the subject is a homozygous carrier of APOE4. In some embodiments, the subject is a heterozygous carrier of MTRES1 rs117058816-G (c.3+1G). In some embodiments, the subject is a homozygous carrier of MTRES1 rs117058816-G (c.3+1G).

In some embodiments, a polygenic risk score is calculated. In some embodiments, the polygenic risk score includes APOE. In some embodiments, the polygenic risk score does not include APOE. In some embodiments, the polygenic risk score includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 variants. In some embodiments, the polygenic risk score includes at least about 100, 200, 300, 400, 500, 600, 700, 800, 900, 1,000, 2,000, 3,000, 4,000, 5,000, 6,000, 7,000, 8,000, 9,000, 10,000 or more variants. In some embodiments, the polygenic risk score includes at least 1000, 10,000, 100,000, 1,000,000 or more variants.

In some embodiments, the steps of calculating a polygenic risk score comprise providing a sample from a subject, optionally purifying DNA from the sample by processing the sample, assaying the optionally processed sample to detect genotypes of at least two genetic loci in the sample, processing the genotypes to produce a polygenic risk score (PRS), calculating the percentile risk of the subject by comparing the PRS to a reference population and selecting a therapy to treat a disease or disorder of the subject based on the percentile.

In some embodiments, a subject is at risk for developing Alzheimer's disease or dementia if the subject has a polygenic risk scope in the upper 50th percentile, 40th percentile, 30th percentile, 20th percentile, 10th percentile, 5th percentile, 4th percentile, 3rd percentile, 2nd percentile, or 1st percentile. In some embodiments, a subject is at risk for developing Alzheimer's disease or dementia if the subject has a polygenic risk score in the upper 20th percentile. In some embodiments, a subject is at risk for developing Alzheimer's disease or dementia if the subject has a polygenic risk score in the upper 40th percentile.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85 (3): 1932-9.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLID sequencing. In some cases, next-generation sequencing involves high-throughput sequencing methods. Additional sequencing methods available to one of skill in the art may also be employed.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

### D. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline cognitive function measurement, a baseline central nervous system (CNS) amyloid plaque measurement, a baseline CNS tau accumulation measurement, a baseline cerebrospinal fluid (CSF) beta-amyloid 42 measurement, a baseline CSF tau measurement, a baseline CSF phospho-tau measurement, a baseline neurofilament light (NfL) measurement. a baseline CSF alpha-synuclein measurement, or a baseline Lewy body measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline cognitive function measurement. The baseline cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The baseline cognitive function measurement may include a score. The baseline cognitive function measurement may be indicative of mild cognitive impairment, or of severe cognitive impairment. The baseline cognitive function measurement may be indicative of a neurological disorder.

The baseline measurement may include a baseline. In some embodiments, the marker of neurodegeneration measurement. Examples of marker of neurodegeneration may include central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein. Any of these measurements may be reduced in relation to the baseline measurement. Some examples of ways to measure these may include an assay such as a immunoassay, colorimetric assay, or microscopy.

In some embodiments, the baseline measurement is a baseline amyloid plaque measurement. The baseline amyloid plaque measurement may include a central nervous system (CNS) amyloid plaque measurement. In some embodiments, the baseline amyloid plaque measurement includes a baseline concentration or amount. The baseline amyloid plaque measurement may be performed using an imaging device. The imaging device may include a positron emission tomography (PET) device. The baseline amyloid plaque measurement may be performed on a biopsy. The baseline amyloid plaque measurement may be performed using a spinal tap (for example, when the baseline amyloid plaque measurement includes a baseline cerebrospinal fluid (CSF) amyloid plaque measurement). In some embodiments, the baseline amyloid plaque measurement is obtained by an assay such as an immunoassay. The baseline beta amyloid plaque measurement may be indicative of a neurodegenerative disease such as Alzheimer's disease.

In some embodiments, the baseline measurement is a baseline beta-amyloid 42 measurement. The baseline beta-amyloid 42 measurement may include a cerebrospinal fluid (CSF) beta-amyloid 42 measurement. In some embodiments, the baseline beta-amyloid 42 measurement includes a baseline concentration or amount. The baseline beta-amyloid 42 measurement may be performed on a biopsy. The baseline beta-amyloid 42 measurement may be performed using a spinal tap (for example, when the baseline beta-amyloid 42 measurement includes a baseline CSF beta-amyloid 42 measurement). In some embodiments, the baseline beta-amyloid 42 measurement is obtained by an assay such as an immunoassay. The baseline beta-amyloid 42 measurement may be indicative of a neurodegenerative disease such as Alzheimer's disease.

In some embodiments, the baseline measurement is a baseline tau measurement. In some embodiments, the baseline tau measurement includes a baseline concentration or amount. The baseline tau measurement may be performed on a biopsy. In some embodiments, the baseline tau measurement is obtained by an assay such as an immunoassay. The baseline beta tau measurement may be indicative of a neurodegenerative disease such as Alzheimer's disease or Parkinson's disease.

In some embodiments, the baseline tau measurement is a baseline central nervous system (CNS) tau measurement. The baseline tau measurement may include a baseline total tau measurement. The baseline tau measurement may include a baseline unphosphorylated tau measurement. The baseline tau measurement may include a baseline phosphorylated tau (phospho-tau) measurement. In some embodiments, the baseline tau measurement is a baseline tau accumulation measurement. In some embodiments, the baseline tau measurement is a baseline CNS tau accumulation measurement. The baseline CNS tau accumulation measurement may be indicative of a neurodegenerative disease such as Alzheimer's disease or Parkinson's disease.

The baseline tau measurement may include a cerebrospinal fluid (CSF) tau measurement. The baseline CSF tau measurement may be performed after use of a spinal tap. The baseline CSF tau measurement may be indicative of a neurodegenerative disease such as Alzheimer's disease or Parkinson's disease.

The baseline CSF tau measurement may include a baseline CSF phospho-tau measurement. The baseline CSF phospho-tau measurement may include an amount of phospho-tau in relation to total tau or unphosphorylated tau. For example, the baseline CSF phospho-tau measurement may include a phospho-tau/tau ratio. The baseline CSF phospho-tau measurement may be indicative of a neurodegenerative disease such as Alzheimer's disease or Parkinson's disease.

In some embodiments, the baseline neurofilament light chain (NfL) measurement includes a baseline CSF or plasma NfL measurement. The baseline NfL measurement may be a baseline CSF NfL measurement. The baseline NfL measurement may be a baseline plasma NfL measurement. The NfL measurement may include a concentration or an amount. The baseline NfL measurement may be indicative of a neurodegenerative disease such as Alzheimer's disease or Parkinson's disease.

In some embodiments, the baseline measurement is a baseline alpha-synuclein measurement. The baseline alpha-synuclein measurement may include a cerebrospinal fluid (CSF) alpha-synuclein measurement. In some embodiments, the baseline alpha-synuclein measurement includes a baseline concentration or amount. The baseline alpha-synuclein measurement may be performed on a biopsy. The baseline alpha-synuclein measurement may be performed using a spinal tap (for example, when the baseline alpha-synuclein measurement includes a baseline CSF alpha-synuclein measurement). In some embodiments, the baseline alpha-synuclein measurement is obtained by an assay such as an immunoassay. The baseline alpha-synuclein measurement may be indicative of a neurodegenerative disease such as Parkinson's disease. The baseline alpha-synuclein measurement may be indicative of dementia.

In some embodiments, the baseline measurement is a baseline Lewy body measurement. The baseline Lewy body measurement may include a central nervous system (CNS) Lewy body measurement. In some embodiments, the baseline Lewy body measurement includes a baseline concentration or amount. The baseline Lewy body measurement may be performed using an imaging device. The imaging device may include a positron emission tomography (PET) device. The baseline beta Lewy body measurement may be indicative of dementia.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. In some embodiments, the fluid sample is a CSF sample. In some embodiments, the fluid sample includes a central nervous system (CNS) fluid sample. The CNS fluid may include cerebrospinal fluid (CSF). In some embodiments, the fluid sample includes a CSF sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample.

In some embodiments, the sample comprises a tissue. In some embodiments, the sample is a tissue sample. In some embodiments, the tissue comprises central nervous system (CNS) tissue. For example, the baseline biomarker measurement, may be obtained in a CNS tissue sample obtained from the patient. The CNS tissue may include brain tissue. The CNS tissue may include nerve tissue. The CNS tissue may include neurons, glia, microglia, astrocytes, or oligodendrocytes, or a combination thereof. The CNS tissue may include neurons. The CNS tissue may include glia. The CNS tissue may include microglia. The CNS tissue may include astrocytes. The CNS tissue may include oligodendrocytes.

In some embodiments, the sample includes cells. In some embodiments, the sample comprises a cell. In some embodiments, the cell comprises a CNS cell. The CNS cell may include a brain cell. The CNS cell may include a nerve cell. The CNS cell may be a neuron, glial cell, microglial cell, astrocyte, or oligodendrocyte. The CNS cell may be a neuron. The CNS cell may be a glial cell. The CNS cell may be a microglial cell. The CNS cell may be an astrocyte. The CNS cell may be an oligodendrocyte.

### E. Effects

In some embodiments, the composition or administration of the composition affects a measurement such as a cognitive function measurement, a central nervous system (CNS) amyloid plaque measurement, a CNS tau accumulation measurement, a cerebrospinal fluid (CSF) beta-amyloid 42 measurement, a CSF tau measurement, a CSF phospho-tau measurement, a NfL measurement, a CSF alpha-synuclein measurement, or a Lewy body measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of a neurological disorder may be reduced upon administration of the composition. The neurological disorder may include dementia, Alzheimer's disease, delirium, cognitive decline, vascular dementia, or Parkinson's disease. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective phenotype of a neurological disorder may be increased upon administration of the composition. The neurological disorder may include dementia, Alzheimer's disease, delirium, cognitive decline, vascular dementia, or Parkinson's disease. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a cognitive function measurement. The cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The cognitive function measurement may include a score. The cognitive function measurement may be indicative of a lack of cognitive impairment. In some embodiments, the cognitive function measurement is indicative of mild cognitive impairment, and the baseline cognitive function measurement is indicative of severe cognitive impairment. The cognitive function measurement may be indicative of a neurological disorder.

In some embodiments, the composition increases the cognitive function measurement relative to the baseline cognitive function measurement. In some embodiments, the increase is measured directly in the subject after administering the composition to the subject. In some embodiments, the cognitive function measurement is increased by about 2.5% or more, about 5% or more, or about 7.5% or more, relative to the baseline cognitive function measurement. In some embodiments, the cognitive function measurement is increased by about 10% or more, relative to the baseline cognitive function measurement. In some embodiments, the cognitive function measurement is increased by about 20% or more, about 30% or more, about 40% or more, about 50% or more, about 60% or more, about 70% or more, about 80% or more, about 90% or more, relative to the baseline cognitive function measurement. In some embodiments, the cognitive function measurement is increased by about 100% or more, increased by about 250% or more, increased by about 500% or more, increased by about 750% or more, or increased by about 1000% or more, relative to the baseline cognitive function measurement. In some embodiments, the cognitive function measurement is increased by no more than about 2.5%, no more than about 5%, or no more than about 7.5%, relative to the baseline cognitive function measurement. In some embodiments, the cognitive function measurement is increased by no more than about 10%, relative to the baseline cognitive function measurement. In some embodiments, the cognitive function measurement is increased by no more than about 20%, no more than about 30%, no more than about 40%, no more than about 50%, no more than about 60%, no more than about 70%, no more than about 80%, no more than about 90%, or no more than about 100% relative to the baseline cognitive function measurement. In some embodiments, the cognitive function measurement is increased by no more than about 100%, increased by no more than about 250%, increased by no more than about 500%, increased by no more than about 750%, or increased by no more than about 1000%, relative to the baseline cognitive function measurement. In some embodiments, the cognitive function measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is an amyloid plaque measurement. The amyloid plaque measurement may include a central nervous system (CNS) amyloid plaque measurement. In some embodiments, the amyloid plaque measurement includes a concentration or amount. The amyloid plaque measurement may be performed using an imaging device. The imaging device may include a positron emission tomography (PET) device. The amyloid plaque measurement may be performed on a biopsy. The amyloid plaque measurement may be performed using a spinal tap (for example, when the amyloid plaque measurement includes a cerebrospinal fluid (CSF) amyloid plaque measurement). In some embodiments, the amyloid plaque measurement is obtained by an assay such as an immunoassay. The beta amyloid plaque measurement may be indicative of a treatment effect of the small molecule on a neurodegenerative disease such as Alzheimer's disease.

In some embodiments, the composition reduces the amyloid plaque measurement relative to the baseline amyloid plaque measurement. In some embodiments, the reduction is measured in a second sample obtained from the subject after administering the composition to the subject. In some embodiments, the reduction is measured directly in the subject after administering the composition to the subject. In some embodiments, the amyloid plaque measurement is decreased by about 2.5% or more, about 5% or more, or about 7.5% or more, relative to the baseline amyloid plaque measurement. In some embodiments, the amyloid plaque measurement is decreased by about 10% or more, relative to the baseline amyloid plaque measurement. In some embodiments, the amyloid plaque measurement is decreased by about 20% or more, about 30% or more, about 40% or more, about 50% or more, about 60% or more, about 70% or more, about 80% or more, about 90% or more, relative to the baseline amyloid plaque measurement. In some embodiments, the amyloid plaque measurement is decreased by no more than about 2.5%, no more than about 5%, or no more than about 7.5%, relative to the baseline amyloid plaque measurement. In some embodiments, the amyloid plaque measurement is decreased by no more than about 10%, relative to the baseline amyloid plaque measurement. In some embodiments, the amyloid plaque measurement is decreased by no more than about 20%, no more than about 30%, no more than about 40%, no more than about 50%, no more than about 60%, no more than about 70%, no more than about 80%, no more than about 90%, or no more than about 100% relative to the baseline amyloid plaque measurement. In some embodiments, the amyloid plaque measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a beta-amyloid 42 measurement. The beta-amyloid 42 measurement may include a cerebrospinal fluid (CSF) beta-amyloid 42 measurement. In some embodiments, the beta-amyloid 42 measurement includes a concentration or amount. The beta-amyloid 42 measurement may be performed on a biopsy. The beta-amyloid 42 measurement may be performed using a spinal tap (for example, when the beta-amyloid 42 measurement includes a CSF beta-amyloid 42 measurement). In some embodiments, the beta-amyloid 42 measurement is obtained by an assay such as an immunoassay. The beta-amyloid 42 measurement may be indicative of a treatment effect of the small molecule on a neurodegenerative disease such as Alzheimer's disease.

In some embodiments, the composition reduces the CSF beta-amyloid 42 measurement relative to the baseline beta-amyloid 42 measurement. In some embodiments, the reduction is measured in a second sample (for example, a CSF sample) obtained from the subject after administering the composition to the subject. In some embodiments, the CSF beta-amyloid 42 measurement is decreased by about 2.5% or more, about 5% or more, or about 7.5% or more, relative to the baseline CSF beta-amyloid 42 measurement. In some embodiments, the CSF beta-amyloid 42 measurement is decreased by about 10% or more, relative to the baseline CSF beta-amyloid 42 measurement. In some embodiments, the CSF beta-amyloid 42 measurement is decreased by about 20% or more, about 30% or more, about 40% or more, about 50% or more, about 60% or more, about 70% or more, about 80% or more, about 90% or more, relative to the baseline CSF beta-amyloid 42 measurement. In some embodiments, the CSF beta-amyloid 42 measurement is decreased by no more than about 2.5%, no more than about 5%, or no more than about 7.5%, relative to the baseline CSF beta-amyloid 42 measurement. In some embodiments, the CSF beta-amyloid 42 measurement is decreased by no more than about 10%, relative to the baseline CSF beta-amyloid 42 measurement. In some embodiments, the CSF beta-amyloid 42 measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF beta-amyloid 42 measurement. In some embodiments, the CSF beta-amyloid 42 measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a tau measurement. In some embodiments, the tau measurement includes a concentration or amount. The tau measurement may be performed on a biopsy. In some embodiments, the tau measurement is obtained by an assay such as an immunoassay. The beta tau measurement may be indicative of a treatment effect of the small molecule on a neurodegenerative disease such as Alzheimer's disease or Parkinson's disease.

In some embodiments, the tau measurement is a central nervous system (CNS) tau measurement. The tau measurement may include a total tau measurement. The tau measurement may include a unphosphorylated tau measurement. The tau measurement may include a phosphorylated tau (phospho-tau) measurement. In some embodiments, the tau measurement is a tau accumulation measurement. In some embodiments, the tau measurement is a CNS tau accumulation measurement. The CNS tau accumulation measurement may be indicative of a treatment effect of the small molecule on a neurodegenerative disease such as Alzheimer's disease or Parkinson's disease.

In some embodiments, the composition reduces the CNS tau accumulation measurement relative to the baseline CNS tau accumulation measurement. In some embodiments, the reduction is measured in a second sample obtained from the subject after administering the composition to the subject. In some embodiments, the CNS tau accumulation measurement is decreased by about 2.5% or more, about 5% or more, or about 7.5% or more, relative to the baseline CNS tau accumulation measurement. In some embodiments, the CNS tau accumulation measurement is decreased by about 10% or more, relative to the baseline CNS tau accumulation measurement. In some embodiments, the CNS tau accumulation measurement is decreased by about 20% or more, about 30% or more, about 40% or more, about 50% or more, about 60% or more, about 70% or more, about 80% or more, about 90% or more, relative to the baseline CNS tau accumulation measurement. In some embodiments, the CNS tau accumulation measurement is decreased by no more than about 2.5%, no more than about 5%, or no more than about 7.5%, relative to the baseline CNS tau accumulation measurement. In some embodiments, the CNS tau accumulation measurement is decreased by no more than about 10%, relative to the baseline CNS tau accumulation measurement. In some embodiments, the CNS tau accumulation measurement is decreased by no more than about 20%, no more than about 30%, no more than about 40%, no more than about 50%, no more than about 60%, no more than about 70%, no more than about 80%, no more than about 90%, or no more than about 100% relative to the baseline CNS tau accumulation measurement. In some embodiments, the CNS tau accumulation measurement is decreased by 2.5%, 5%, 7.5%, 10%, 20%, 30%, 40%, 50%, 60%, 70%, 80%, 90%, or 100%, or by a range defined by any of the two aforementioned percentages.

The tau measurement may include a cerebrospinal fluid (CSF) tau measurement. The CSF tau measurement may be performed after use of a spinal tap. The CSF tau measurement may be indicative of a treatment effect of the small molecule on a neurodegenerative disease such as Alzheimer's disease or Parkinson's disease.

In some embodiments, the composition reduces the CSF tau measurement relative to the baseline CSF tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF tau measurement is decreased by about 2.5% or more, about 5% or more, or about 7.5% or more, relative to the baseline CSF tau measurement. In some embodiments, the CSF tau measurement is decreased by about 10% or more, relative to the baseline CSF tau measurement. In some embodiments, the CSF tau measurement is decreased by about 20% or more, about 30% or more, about 40% or more, about 50% or more, about 60% or more, about 70% or more, about 80% or more, about 90% or more, relative to the baseline CSF tau measurement. In some embodiments, the CSF tau measurement is decreased by no more than about 2.5%, no more than about 5%, or no more than about 7.5%, relative to the baseline CSF tau measurement. In some embodiments, the CSF tau measurement is decreased by no more than about 10%, relative to the baseline CSF tau measurement. In some embodiments, the CSF tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF tau measurement. In some embodiments, the CSF tau measurement is decreased by 2.5%, 5%, 7.5%, 10%, 20%, 30%, 40%, 50%, 60%, 70%, 80%, 90%, or 100%, or by a range defined by any of the two aforementioned percentages.

The CSF tau measurement may include a CSF phospho-tau measurement. The CSF phospho-tau measurement may include an amount of phospho-tau in relation to total tau or unphosphorylated tau. For example, the CSF phospho-tau measurement may include a phospho-tau/tau ratio. The CSF phospho-tau measurement may be indicative of a treatment effect of the small molecule on a neurodegenerative disease such as Alzheimer's disease or Parkinson's disease.

In some embodiments, the composition reduces the CSF phospho-tau measurement relative to the baseline CSF phospho-tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF phospho-tau measurement is decreased by about 2.5% or more, about 5% or more, or about 7.5% or more, relative to the baseline CSF phospho-tau measurement. In some embodiments, the CSF phospho-tau measurement is decreased by about 10% or more, relative to the baseline CSF phospho-tau measurement. In some embodiments, the CSF phospho-tau measurement is decreased by about 20% or more, about 30% or more, about 40% or more, about 50% or more, about 60% or more, about 70% or more, about 80% or more, about 90% or more, relative to the baseline CSF phospho-tau measurement. In some embodiments, the CSF phospho-tau measurement is decreased by no more than about 2.5%, no more than about 5%, or no more than about 7.5%, relative to the baseline CSF phospho-tau measurement. In some embodiments, the CSF phospho-tau measurement is decreased by no more than about 10%, relative to the baseline CSF phospho-tau measurement. In some embodiments, the CSF phospho-tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF phospho-tau measurement. In some embodiments, the CSF phospho-tau measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the neurofilament light chain (NfL) measurement includes a CSF or plasma NfL measurement. The NfL measurement may be a CSF NfL measurement. The NfL measurement may be a plasma NfL measurement. The NfL measurement may include a concentration or an amount. The NfL measurement may be indicative of a neurodegenerative disease such as Alzheimer's disease or Parkinson's disease.

In some embodiments, the composition reduces the NfL measurement relative to the baseline NfL measurement. In some embodiments, the reduction is measured in a second sample obtained from the subject after administering the composition to the subject. In some embodiments, the NfL measurement is decreased by about 2.5% or more, about 5% or more, or about 7.5% or more, relative to the baseline NfL measurement. In some embodiments, the NfL measurement is decreased by about 10% or more, relative to the baseline NfL measurement. In some embodiments, the NfL measurement is decreased by about 20% or more, about 30% or more, about 40% or more, about 50% or more, about 60% or more, about 70% or more, about 80% or more, about 90% or more, relative to the baseline NfL measurement. In some embodiments, the NfL measurement is decreased by no more than about 2.5%, no more than about 5%, or no more than about 7.5%, relative to the baseline NfL measurement. In some embodiments, the NfL measurement is decreased by no more than about 10%, relative to the baseline NfL measurement. In some embodiments, the NfL measurement is decreased by no more than about 20%, no more than about 30%, no more than about 40%, no more than about 50%, no more than about 60%, no more than about 70%, no more than about 80%, no more than about 90%, or no more than about 100% relative to the baseline NfL measurement. In some embodiments, the NfL measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a alpha-synuclein measurement. The alpha-synuclein measurement may include a cerebrospinal fluid (CSF) alpha-synuclein measurement. In some embodiments, the alpha-synuclein measurement includes a concentration or amount. The alpha-synuclein measurement may be performed on a biopsy. The alpha-synuclein measurement may be performed using a spinal tap (for example, when the alpha-synuclein measurement includes a CSF alpha-synuclein measurement). In some embodiments, the alpha-synuclein measurement is obtained by an assay such as an immunoassay. The alpha-synuclein measurement may be indicative of a treatment effect of the small molecule on a neurodegenerative disease such as Parkinson's disease. The alpha-synuclein measurement may be indicative of a treatment effect of the small molecule on dementia.

In some embodiments, the composition reduces the alpha-synuclein measurement relative to the baseline alpha-synuclein measurement. In some embodiments, the reduction is measured in a second sample obtained from the subject after administering the composition to the subject. In some embodiments, the alpha-synuclein measurement is decreased by about 2.5% or more, about 5% or more, or about 7.5% or more, relative to the baseline alpha-synuclein measurement. In some embodiments, the alpha-synuclein measurement is decreased by about 10% or more, relative to the baseline alpha-synuclein measurement. In some embodiments, the alpha-synuclein measurement is decreased by about 20% or more, about 30% or more, about 40% or more, about 50% or more, about 60% or more, about 70% or more, about 80% or more, about 90% or more, relative to the baseline alpha-synuclein measurement. In some embodiments, the alpha-synuclein measurement is decreased by no more than about 2.5%, no more than about 5%, or no more than about 7.5%, relative to the baseline alpha-synuclein measurement. In some embodiments, the alpha-synuclein measurement is decreased by no more than about 10%, relative to the baseline alpha-synuclein measurement. In some embodiments, the alpha-synuclein measurement is decreased by no more than about 20%, no more than about 30%, no more than about 40%, no more than about 50%, no more than about 60%, no more than about 70%, no more than about 80%, no more than about 90%, or no more than about 100% relative to the baseline alpha-synuclein measurement. In some embodiments, the alpha-synuclein measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a Lewy body measurement. The Lewy body measurement may include a central nervous system (CNS) Lewy body measurement. In some embodiments, the Lewy body measurement includes a concentration or amount. The Lewy body measurement may be performed using an imaging device. The imaging device may include a positron emission tomography (PET) device. The beta Lewy body measurement may be indicative of a treatment effect of the small molecule on dementia.

In some embodiments, the composition reduces the Lewy body measurement relative to the baseline Lewy body measurement. In some embodiments, the reduction is measured in a second sample obtained from the subject after administering the composition to the subject. In some embodiments, the reduction is measured directly in the subject after administering the composition to the subject. In some embodiments, the Lewy body measurement is decreased by about 2.5% or more, about 5% or more, or about 7.5% or more, relative to the baseline Lewy body measurement. In some embodiments, the Lewy body measurement is decreased by about 10% or more, relative to the baseline Lewy body measurement. In some embodiments, the Lewy body measurement is decreased by about 20% or more, about 30% or more, about 40% or more, about 50% or more, about 60% or more, about 70% or more, about 80% or more, about 90% or more, relative to the baseline Lewy body measurement. In some embodiments, the Lewy body measurement is decreased by no more than about 2.5%, no more than about 5%, or no more than about 7.5%, relative to the baseline Lewy body measurement. In some embodiments, the Lewy body measurement is decreased by no more than about 10%, relative to the baseline Lewy body measurement. In some embodiments, the Lewy body measurement is decreased by no more than about 20%, no more than about 30%, no more than about 40%, no more than about 50%, no more than about 60%, no more than about 70%, no more than about 80%, no more than about 90%, or no more than about 100% relative to the baseline Lewy body measurement. In some embodiments, the Lewy body measurement is decreased by 2.5%, 5%, 7.5%, 10%, 20%, 30%, 40%, 50%, 60%, 70%, 80%, 90%, or 100%, or by a range defined by any of the two aforementioned percentages.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7] cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (—O), thioxo (═S), cyano (—CN), nitro (—NO₂), imino (═N—H), oximo (═N—OH), hydrazino (═N—NH₂), —R^{b}OR^{a}, —R^{b}OC(O)R^{a}, —R^{b}OC(O)OR^{a}, —R^{b}OC(O)N(R^{a})₂, —R^{b}N(R^{a})₂, —R^{b}C(O)R^{a}, —R^{b}C(O)OR^{a}, —R^{b}C(O)N(R^{a})₂, —R^{b}OR^{b}C(O)N(R^{a})₂, —R^{b}N(R^{a})C(O)OR^{a}, —R^{b}N(R^{a})C(O)R^{a}, —R^{b}(R^{a})S(O)ₜR^{a} (where t is 1 or 2), —R^{b}(O)ₜR^{a} (where t is 1 or 2), —R^{b}S(O)ₜOR^{a} (where t is 1 or 2), and —R^{b}S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}OR^{a}, —R^{b}OC(O)R^{a}, —R^{b}OC(O)OR^{a}, —R^{b}OC(O)N(R^{a})₂, —R^{b}N(R^{a})₂, —R^{b}C(O)R^{a}, —R^{b}C(O)OR^{a}, —R^{b}C(O)N(R^{a})₂, —R^{b}OR^{b}C(O)N(R^{a})₂, —R^{b}N(R^{a})C(O)OR^{a}, —R^{b}N(R^{a})C(O)R^{a}, —R^{b}N(R^{a})S(O)ₜR^{a} (where t is 1 or 2), —R^{b}S(O)ₜR^{a} (where t is 1 or 2), —R^{b}S(O)ₜOR^{a} (where t is 1 or 2) and —R^{b}S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (—O), thioxo (═S), cyano (—CN), nitro (—NO₂), imino (═N—H), oximo (═N—OH), hydrazine (═N—NH₂), —R^{b}OR^{a}, —R^{b}OC(O)R^{a}, —R^{b}OC(O)OR^{a}, —R^{b}OC(O)N(R^{a})₂, —R^{b}N(R^{a})₂, —R^{b}C(O)R^{a}, —R^{b}C(O)OR^{a}, —R^{b}C(O)N(R^{a})₂, —R^{b}OR^{b}C(O)N(R^{a})₂, —R^{b}N(R^{a})C(O)OR^{a}, —R^{b}N(R^{a})C(O)R^{a}, —R^{b}N(R^{a})S(O)ₜR^{a} (where t is 1 or 2), —R^{b}S(O)ₜR^{a} (where tis 1 or 2), —R^{b}S(O)ₜOR^{a} (where t is 1 or 2) and —R^{b}S(O)ₜN(R^{a})₂ (where tis 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

The section headings used herein are for organizational purposes only and are not to be construed as limiting the subject matter described.

### VI. Examples

### Example 1: A Splice Donor Variant in MTRES1 Demonstrates Protective Associations for Dementia and Alzheimer's Disease Related Traits

Variants in MTRES1 were evaluated for associations with dementia, Alzheimer's disease and related traits in approximately 452,000 individuals with genotype data from the UK Biobank cohort. rs117058816 is a rare (AAF=0.006) splice donor variant (c.3+1G>A) in MTRES1. This variant is considered to be a loss of function variant that results in a loss or change in the abundance or activity of the MTRES1 gene product.

The analyses resulted in identification of dementia and Alzheimer's disease-related associations for the MTRES1 c.3+1G>A variant. For example, c.3+1G>A was associated with decreased risk of Alzheimer's disease, dementia, delirium, and vascular dementia. c.3+1G>A was also associated with decreased risk of family history of Alzheimer's disease and decreased risk of dementia medication use (Table 1A and 1B).

TABLE 1A
MTRES1 Dementia, Alzheimer's and related trait associations

| | | | | | | Family History of | |
|---|---|---|---|---|---|---|---|
| | | | | Alzheimer's Disease | | Alzheimer's Disease | |
| | | | | (n = 2,864) | | (n = 53,344) | |
| Variant | Gene | Function | AAF | P value | OR | P value | OR |
| rs117058816 | MTRES1 | Splice donor; c.3 + 1G > A | 0.006 | 2.58E−04 | ↓0.459 | 9.54E−03 | ↓0.893 |

TABLE 1B
MTRES1 Dementia, Alzheimer's and related trait associations

| | Dementia | | Anticholinesterase | | Delirium | | Vascular Dementia | |
|---|---|---|---|---|---|---|---|---|
| | (n = 4,009) | | Medication (n = 813) | | (n = 3,901) | | (n = 807) | |
| Variant | P value | OR | P value | OR | P value | OR | P value | OR |
| rs117058816 | 7.92E−07 | ↓0.489 | 8.04E−03 | ↓0.613 | 7.75E−03 | ↓0.667 | 7.44E−04 | ↓0.208 |

These results indicate that loss or change of function of MTRES1 results in protection from dementia and Alzheimer's disease and related diseases. These results further indicate that therapeutic modulation of MTRES1 may result in similar disease-protective effects.

Minigene Constructs Expressing Protective variants in MTRES1 result in a reduction of MTRES1 mRNA and MTRES1 protein Minigene expression constructs encoding for wild type and rs117058816 (c.3+1G>A) MTRES1 proteins were generated. Minigene constructs (<10 kb) are easier to synthesize and have greater transfection efficiency in downstream experiments than constructs that exceed 10 kb in length. The minigene constructs have a portion of internal, intronic sequence removed, but retain all exons and UTRs. Therefore, the pre-mRNA of the exons, reduced introns, and 5′ and 3′ UTRs of the protein coding transcript (ENST00000625458) of MTRES1 was cloned into a pcDNA3.1 (+) vector driven by a CMV promoter. Empty vector was used as control. For rs117058816 expression constructs, the A allele replaced the G allele at DNA sequence position chr6: 107030108 (human genome build 38). This leads to the loss of a splice donor site (c.3+1G>A).

Transfections of HEK-293 cells were optimized. HEK-293 cells were plated in a 6-well plate in complete growth media and grown for 48 hours followed by a media change. Cells were then transfected with 2 μg of plasmid DNA and 7 μl of TransIT-2020. Cells were incubated for 48 hours, and then harvested.

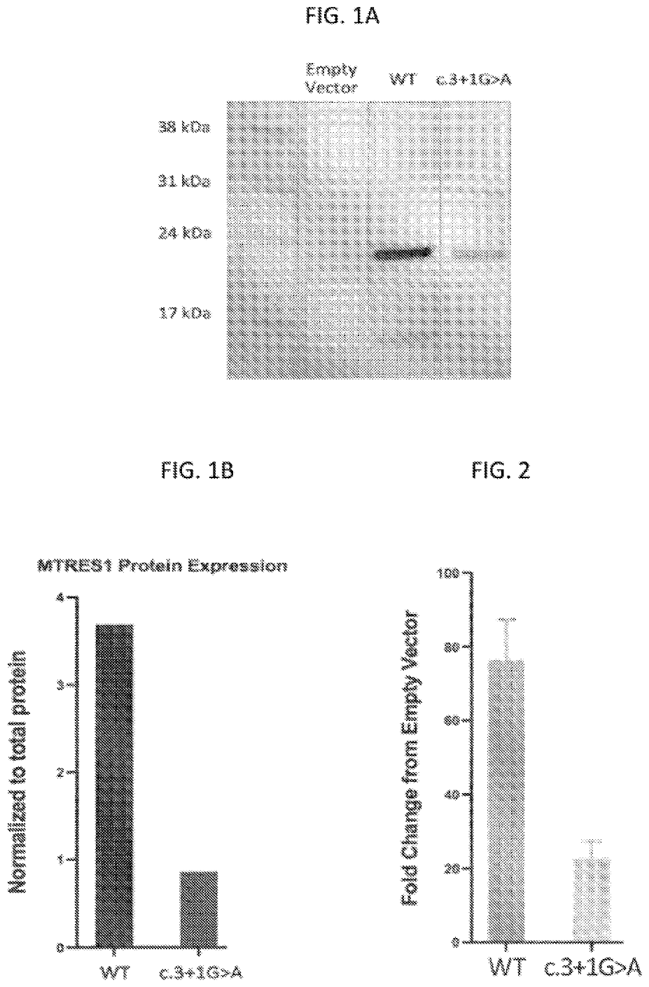

Cell lysates from transfected cells were assayed to evaluate intracellular MTRES1 protein by western blot (FIG. 1A). In empty vector transfected HEK-293 cells, a faint band representing endogenous MTRES1 expression was detected by western blot as a band at 24 kDa. In cells transfected with the wild type construct, significant expression of MTRES1 was detected by western blot as a band at 24 kDa. In cells transfected with the rs117058816 construct, reduced MTRES1 protein compared with wild type was detected by western blot as a band between 24 kDa. When normalizing to total protein, cells transfected with the rs117058816 construct express approximately 75% less MTRES1 protein compared with cells transfected with the wild type construct (FIG. 1B).

Cell lysates from transfected cells were also assayed to evaluate MTRES1 mRNA by qPCR. Cells transfected with the rs117058816 construct express approximately 70% less MTRES1 mRNA compared with cells transfected with the wild type construct (FIG. 2).

These data provide experimental verification that MTRES1 gene variants associated with protection from dementia and Alzheimer's disease result in a change or loss of MTRES1 protein and MTRES1 mRNA abundance or function. Accordingly, in some cases therapeutic modulation or inhibition of MTRES1 may be an effective genetically-informed method of treatment for these diseases.

CRISPR-Engineered Cell Lines Expressing Protective Variants in MTRES1 Result in a Reduction of MTRES1 mRNA and MTRES1 Protein

Sanger-verified CRISPR cell lines on a HEK-293 clonal background were generated including (i) a rs117058816-A (c.3+1A) homozygous knock-in (KI), (ii) a homozygous knock-out (KO) and (iii) a mock-transfected wild type (WT) cell line.

Cells were grown, fractionated and whole cell lysates and mitochondrial fractions harvested

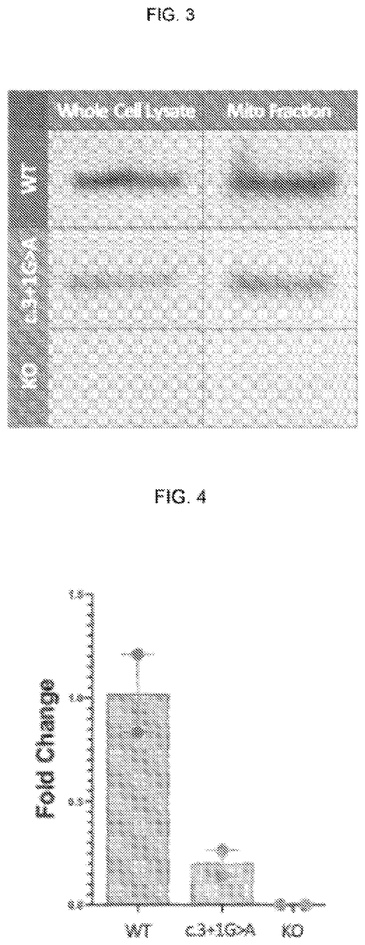

Whole cell and mitochondrial fraction lysates from CRISPR cell lines were assayed to evaluate intracellular MTRES1 protein by western blot (FIG. 3). In WT cells, significant MTRES1 expression was detected by western blot as a band at 24 kDa in both whole cell lysates and mitochondrial fractions. In CRISPR KI cells, reduced MTRES1 protein compared with WT was detected by western blot in both whole cell lysates and mitochondrial fractions. In CRISPR KO cells, no MTRES1 protein was detected by western blot in either whole cell lysates or mitochondrial fractions.

Whole cell lysates from CRISPR cell lines were also assayed to evaluate MTRES1 mRNA by qPCR (FIG. 4). In CRISPR KI cells, approximately 70% less MTRES1 mRNA expression is observed compared with cells transfected with the wild type construct. In CRISPR KO cells, no MTRES1 mRNA expression was detected by qPCR.

These data provide further experimental verification that MTRES1 gene variants associated with protection from dementia and Alzheimer's disease result in change or loss of MTRES1 protein and MTRES1 mRNA abundance or function. Accordingly, in some cases therapeutic modulation or inhibition of MTRES1 may be an effective genetically-informed method of treatment for these diseases.

### Example 2: Modulation of MTRES1 in a Mouse Model for Alzheimer's Disease Using a Small Molecule that Targets MTRES1

In this experiment, a mouse model of Alzheimer's Disease (AD) will be used to evaluate effects of small molecules that target MTRES1. The model includes Tg2576 mice which express human amyloid beta precursor protein (APP) and presenilin-1 (PSEN1) transgenes with five AD-linked mutations. Cognitive function is measured using a forced swimming test (FST).

Seven-month-old mice are divided into two groups: Group 1—a group treated with the small molecule inhibitor of MTRES1, Group 2—a group treated with vehicle.

Mice are administered the small molecule or the vehicle on day 0 of treatment. Every other week thereafter animals from each group will be dosed for a total of 4 injections. The behavioral tests are performed 24 hours after the final injection.

To rule out nonspecific motor effects that could influence the FST results, the potential effect of treatment on locomotor activity is assessed. Mice are evaluated using the openfield paradigm (44×44×40 cm) in a sound-attenuated room. The total distance (cm) traveled by each mouse is recorded for 5 min by a video surveillance system (SMART; Panlab SL, Barcelona, Spain) and is used to quantify activity levels. The floor of the open-field apparatus is cleaned with 10% ethanol between tests.

The FST includes a behavioral test useful for screening potential drugs that influence cognition and assessing other manipulations that are expected to affect cognitive related behaviors. On the first day, mice are placed individually in the water and allowed to swim for 15 min. The next day, mice are placed again in the water to observe the duration of immobility for 6 min using a camera. Following a 1-min session of acclimation to the apparatus, all behaviors are recorded for 5 min by a video surveillance system (SMART 2.5.21; Panlab SL). Immobility is defined as motionless floating in the water, only allowing movements necessary for the animal to keep its head above the water. The total immobility time in the FST is recorded as an index of cognitive ability.

Twenty four hours after the behavioral assessment, the mice are sacrificed by cervical dislocation following an intraperitoneal injection of 0.3 ml Nembutal (5 mg/ml) (Sigma Cat. No. 1507002). Brain and spinal cord tissues are removed and placed in RNAlater for mRNA isolation.

### Example 3: Protective Variants in MTRES1 Modulate APOE E4-Mediated Risk of Alzheimer's Disease and Dementia

The major common genetic risk factor for Alzheimer's disease is the APOE haplotype, with permutations of 2 genetic variants, rs429358 (p.Cys112Arg) and rs7412 (p.Arg158Cys) defining 3 major APOE haplotypes—E2, E3 and E4 (Table 2). The APOE2 haplotype is considered protective, APOE3 haplotype is considered ‘neutral’ and APOE4 haplotype is considered risk for Alzheimer's disease and dementia.

TABLE 2
APOE alleles and haplotypes

| APOE Allele | ε2 | ε3 | ε4 |
|---|---|---|---|
| Haplotype | rs429358-T | rs429358-T | rs429358-C |
| | (p.112Cys) | (p.112Cys) | (p.112Arg) |
| | rs7412-T | rs7412-C | rs7412-C |
| | (p.158Cys) | (p.158Arg) | (p.158Arg) |

Stratified genetic analyses were performed to evaluate the effect of the MTRES1 rs117058816 splice donor variant (c.3+1G>A) on an APOE4 risk background in 452,401 individuals with genotype data from the UK Biobank cohort. All stratified analyses were performed under the assumption that the AP (E3 haplotype and the MTRES1 rs117058816 (c.3+1G>A) reference (G) alleles are ‘neutral’, therefore the reference group in these analyses consists of individuals who are MTRES1 rs117058816 (c.3+1G>A) G/G and APOE E3/E3.

In addition to replicating the well-known association between APOE4 and significantly increased risk of dementia, the analyses indicate that APOE4 homozygous individuals that carry a single copy of MTRES1 rs117058816-A (c.3+1A) alternative (A) allele have approximately half the relative risk of dementia compared with APOE4 homozygous individuals that are homozygous for the MTRES1 rs117058816-G (c.3+1G) reference (G) allele (Table 3).

TABLE 3
APOE4 and MTRES1 rs117058816 (c.3 +
1G > A) stratified analyses

| | | | Dementia (n = 4,009) | | |
|---|---|---|---|---|---|
| | | | | | APOE4/E4 |
| | | | | | Relative |
| Variant | Gene | EAF | P value | OR | Risk |
| E4/E4 vs. E3/E3 | APOE | 0.02 | <5E−324 | ↑10.688 | 1.00 |
| G/G E4/E4 vs. | MTRES1/ | 0.02 | <5E−324 | ↑10.652 | 0.997 |
| G/G E3/E3 | APOE | | | | |
| G/A E4/E4 vs. | MTRES1/ | 0.003 | 0.003 | ↑5.412 | ↓0.506 |
| G/G E3/E3 | APOE | | | | |

These results indicate that change or loss of function of MTRES1 results in protection from dementia even in the context of the exceptionally high genetic risk conferred by APOE4 homozygosity. These results further indicate that therapeutic modulation or inhibition of MTRES1 may result in similar disease-protective effects in APOE4 heterozygous and homozygous carriers at increased genetic risk of Alzheimer's disease and dementia.

### Example 4: Protective Variants in MTRES1 Modulate Polygenic Risk of Alzheimer's Disease and Dementia

Polygenic risk scores (PRS) are increasingly being utilized in the screening, diagnosis and treatment of disease. PRS aggregate the effects (or weights) of a large number of genetic variants on a given disease to estimate an individual's risk for that disease. Those individuals within the highest percentiles of the score tend to have a higher incidence of the disease as compared to individuals in the percentiles below, and this information can be utilized in both clinical and research settings to select individuals most likely to benefit from a diagnostic test or therapeutic intervention.

A PRS score was generated using publicly-available weights from 37 independent genetic variants, with a p value less than 1e-5 within a meta-analysis of Alzheimer's disease (PGS catalogue-PGS000898). Notably, this set of variants did not include the APOE locus, allowing examination of APOE-independent polygenic risk for Alzheimer's. The PRS score was calculated for 452,401 individuals with genotype data from the UK Biobank cohort, multiplying weights by the number of alternative alleles, and summing across all variants per individual. Examining model performance, individuals in the upper 20^{th} percentile of PRS scores were 1.68 times more likely to have all cause dementia as compared to those in the remaining 80% of samples.

Genetic analyses were performed to evaluate the effect of the MTRES1 rs117058816 splice donor variant (c.3+1G>A) on the risk of dementia in the full sample of 452,401 UK Biobank participants and in subsetted strata comprising the upper 20th and 40th percentiles of the Alzheimer's disease PRS (90,492 participants and 158,331 participants respectively).

The analyses indicate that the MTRES1 rs117058816 splice donor variant (c.3+1G>A) is associated with protection from dementia in all individuals and in individuals at high polygenic risk of Alzheimer's disease, with a notable increase in the already large protective effect size as the polygenic risk increases (allelic odds ratios of 0.489, 0.337, and 0.291 among all individuals and individuals in the top 40th and 20th percentiles for PRS risk respectively) (Table 4).

TABLE 4
MTRES1 rs117058816 (c.3 + 1G > A) associations within Alzheimer's Disease PRS strata

| | | | | Dementia (n = 4,009) | |
|---|---|---|---|---|---|
| Variant | Gene | PRS Strata | N in Strata | P value | OR |
| rs117058816 (c.3 + 1G > A) | MTRES1 | None (all individuals) | 452,401 | 7.92E−07 | ↓0.489 |
| rs117058816 (c.3 + 1G > A) | MTRES1 | Upper 40^{th} Percentile | 158,331 | 7.07E−07 | ↓0.337 |
| rs117058816 (c.3 + 1G > A) | MTRES1 | Upper 20^{th} Percentile | 90,492 | 3.14E−05 | ↓0.291 |

These results indicate that in the context of APOE-independent, high polygenic risk for Alzheimer's disease, MTRES1 change or loss of function confers protection from dementia. These results further indicate that therapeutic modulation or inhibition of MTRES1 may result in similar disease-protective effects in individuals with high polygenic risk for Alzheimer's disease and dementia.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

What is claimed is:

1. A composition comprising a small molecule that targets MTRES1 and when administered to a subject in an effective amount modulates the activity, function, or binding of central nervous system (CNS) MTRES1.

2. The composition of claim 1, wherein the activity, function or binding of CNS MTRES1 is inhibited.

3. The composition of claim 1, wherein the activity, function, or binding of CNS MTRES1 is inhibited by about 10% or more, as compared to prior to administration.

4. A composition comprising a small molecule that targets MTRES1 and when administered to a subject in an effective amount increases cognitive function or slows cognitive decline.

5. The composition of claim 4, wherein the cognitive function is increased by about 10% or more, as compared to prior to administration.

6. The composition of claim 4, wherein the cognitive decline is slowed by about 10% or more, as compared to prior to administration.

7. A composition comprising a small molecule that targets MTRES1 and when administered to a subject in an effective amount decreases a marker of neurodegeneration.

8. The composition of claim 7, wherein the marker of neurodegeneration comprises a central nervous system (CNS) or cerebrospinal fluid (CSF) marker of neurodegeneration.

9. The composition of claim 7, wherein the marker of neurodegeneration comprises a measurement of central nervous system (CNS) amyloid plaques, CNS tau accumulation, cerebrospinal fluid (CSF) beta-amyloid 42, CSF tau, CSF phospho-tau, CSF or plasma neurofilament light chain (NfL), Lewy bodies, or CSF alpha-synuclein.

10. The composition of any one of claims 7-9, wherein the marker of neurodegeneration is decreased by about 10% or more, as compared to prior to administration.

11. A method of treating a subject having a neurological disorder, comprising administering an effective amount of the composition of any one of claims 1-10 to the subject.

12. The method of claim 11, wherein the neurological disorder comprises dementia, Alzheimer's disease, delirium, cognitive decline, vascular dementia, or Parkinson's disease.

13. A method of treating a subject having a neurological disorder or who is at risk for developing the neurological disorder, the method comprising evaluating a subject's risk for developing a neurological disorder and administering an effective amount of the composition of any one of claims 1-10 to the subject.

14. The method of claim 12 or 13, wherein the subject has a genotype at risk for developing Alzheimer's disease or dementia.

15. The method of claim 14, wherein the subject is a heterozygous or homozygous carrier of APOE4.

16. The method of claim 14, wherein the subject is a heterozygous or homozygous carrier of MTRES1 rs117058816-G (c.3+1G).

17. The method of claim 12 or 13, wherein evaluating a subject's risk for developing a neurological disorder comprises calculating a polygenic risk score for developing Alzheimer's disease or dementia.

18. The method of claim 17, wherein the subject has a polygenic risk score in the 40^{th} percentile or higher, which is indicative of a high risk for developing Alzheimer's disease or dementia.

19. The method of claim 17, wherein the subject has a polygenic risk score in the 20^{th} percentile or higher, which is indicative of a high risk for developing Alzheimer's disease or dementia.

20. The method of claim 17, wherein calculating a polygenic risk score comprises providing genomic data comprising one or more genotypes of the subject, wherein the one or more genotypes is associated with a high risk for developing Alzheimer's disease or dementia.
